(19) United States
(12) Patent Application Publication
(10) Pub. No.: US 2025/0025390 A1
(43) Pub. Date: Jan. 23, 2025

(54) AQUEOUS CARE AND/OR MAKEUP COMPOSITION COMPRISING A FATTY ACID MONOESTER, A NEUTRALIZED ANIONIC SURFACTANT, A VP/EICOSENE COPOLYMER AND A DYESTUFF

(71) Applicant: L'OREAL, Paris (FR)

(72) Inventors: Véronique FERRARI, Chevilly Larue (FR); Carole LEMERRER, Chevilly Larue (FR)

(73) Assignee: L'OREAL, Paris (FR)

(21) Appl. No.: 18/713,833

(22) PCT Filed: Nov. 18, 2022
(86) PCT No.: PCT/EP2022/082419

§ 371 Date: May 28, 2024

(30) Foreign Application Priority Data
Nov. 30, 2021 (FR) FR2112737

(51) Int. Cl. A61K 8/37; A61K 8/36; A61K 8/81; A61Q 1/10

(52) U.S. Cl. CPC A61K 8/37; A61K 8/361; A61K 8/8182; A61Q 1/10; A61K 2800/43

(57) ABSTRACT

The present application relates to a composition comprising, notably in a physiologically acceptable medium: (1) at least one linear fatty acid monoester of formula (I) below: [Chem 1] R₁—O—R₂ (I) in which R₁ and R₂ are linear and saturated and have, independently of each other, a number of carbon atoms greater than or equal to 20, with R₁ representing an acyl radical, and R₂ representing an alkyl radical, and (2) at least one anionic surfactant chosen from: —C₁₂-C₂₀ monoalkyl phosphates: —fatty acids containing from 14 to less than 20 carbon atoms: —mixtures thereof, and (3) at least one base that is capable of partially or totally neutralizing said anionic surfactant (2), (4) at least one vinylpyrrolidone/eicosene copolymer, and (5) at least one dyestuff, and (6) water, said composition comprising less than 5.0% by weight of wax(es) relative to the total weight of the composition, preferably less than 3% by weight of wax(es), and more particularly is free of wax.

### TECHNICAL FIELD

The present invention relates to the field of caring for and/or making up keratin materials, and is directed toward proposing compositions more particularly intended for making up the eyelashes or the eyebrows.

In general, compositions intended for making up keratin fibers, for example the eyelashes, aim to densify the thickness and the visual perception of the eyelashes and ultimately the look. These mascaras are described as aqueous or cream mascaras when they are formulated in an aqueous base, and anhydrous mascaras when they are formulated as a dispersion in an organic solvent medium.

A great diversity of cosmetic effects may be afforded by applying a mascara to keratin fibers and notably the eyelashes, for instance a volumizing, lengthening, thickening and more particularly charging makeup effect.

These effects are mostly adjusted through the amount and nature of the particles and most particularly those of the waxes present in the mascaras. In general, mascaras in fact have a significant amount of wax(es) and notably from 10% to 35% by weight of waxes, more generally from 15% to 30% by weight, relative to the total weight thereof.

For obvious reasons, improving the textures of mascara which condition the manifestation of one or more makeup effects is a constant preoccupation of cosmetic formulators.

Moreover, the specific effects associated with a particular formulation, for example charging and moreover providing excellent separation of the made-up eyelashes, are expected to be reproduced virtually identically by all the production batches of one and the same formulation.

In order to meet these expectations and/or objectives, it is therefore necessary to be capable of precisely adjusting the texture of a mascara and of reproducing it as faithfully as possible with batches that are not necessarily manufactured at the same time but which are identical in terms of ingredients and which thus need to afford makeup effects that are in theory also identical.

### PRIOR ART

However, as specified above, most of the mascaras currently available are formulated with a significant amount of waxes. In point of fact, as detailed in Ullmann's Encyclopedia of Industrial Chemistry 2015, Wiley-VCH Verlag GmbH & Co. KGaA, Weinheim 10.1002/14356007.a28.pub2, most waxes do not consist of a single chemical compound, but are instead complex mixtures. They may be mixtures of oligomers and/or of polymers which, in many cases, also have varied molar masses, varied molar mass distributions and also varied degrees of branching. Thus, a polar wax is conventionally made up of a mixture of alkanes, fatty alcohols and fatty esters, the fatty-chain length of which varies according to the melting point.

It is thus very difficult for the manufacturers of these waxes to guarantee a rigorously identical composition for all production batches. More specifically, there may be, between several production batches of one and the same wax, a variability in terms of the chemical nature of some of its constituent compounds. Similarly, the proportionality of some of its constituent compounds may also vary between production batches.

For obvious reasons, these variabilities have a non-negligible impact on the properties of the wax and thus on those of the mascara incorporating this wax in significant amount. Thus, two mascara formulations of identical composition and therefore produced from one and the same conventional wax and in the same amount, can nevertheless differ in terms of rheological properties and therefore of texture and mechanical properties of the deposition of the product on keratin fibers, such as resistance to flaking, if they were produced from two separate production batches of this wax.

Consequently, the use of conventional waxes, in particular in significant amount, in mascara compositions does not make it possible to guarantee for users the reproduction of the finely adjusted and totally identical rheological properties and mechanical properties of the deposition of the product on keratin fibers, such as resistance to flaking, in all the mascara specimens of one and the same composition.

In patent applications FR3080034, EP3781118, EP3897859, EP3897558 and EP3897519, aqueous compositions, notably mascaras, were proposed comprising at least one fatty acid monoester such as behenyl behenate, at least one fatty acid containing from 14 carbon atoms to less than 20 carbon atoms, at least one base that is capable of at least partially neutralizing said fatty acid and pigments, notably iron oxides. However, these compositions are not entirely satisfactory in terms of the stability of the intensity of the color over time, notably over several days and according to temperature variations.

The example 2 of mascara containing behenyl behenate, 4.5% by weight of Candelilla wax and behenic acid (C22) is also known from patent application US2008292668.

Patent application US 2015/174056 proposed a cosmetic composition for coating keratin fibers of the emulsion type, including:
an aqueous phase, at least one wax, an emulsifying system, at least one film-forming polymer present in a dry matter content of greater than or equal to 5% by weight relative to the total weight of the composition, at least one organic, preferably polymeric, lipophilic gelling agent, in which the wax(es) and the emulsifying system are present in a respective total content such that the weight ratio of the wax(es) to the emulsifying system is less than or equal to 1. Examples are compositions comprising 5.5% of waxes, a vinylpyrrolidone/eicosene copolymer, a pigment, and stearic acid neutralized with triethanolamine.

The commercial product Super Drama Volume and Strengthening Waterproof mascara Mintel ID 2600879 comprising water, a large amount of waxes, behenyl behenate, stearic acid neutralized with an organic base, vinylpyrrolidone/eicosene copolymer and dyestuffs is also known.

Finally, aqueous compositions based on semicrystalline polymer, an silicone elastomer gel and a hydrophilic gelling agent of the ammonium polyacryloyldimethyl taurate type were proposed in patent U.S. Pat. No. 8,932,573. Examples describe mascaras with vinylpyrrolidone/eicosene copolymer, stearic acid, large amounts of conventional waxes and iron oxides.

Thus, a first aim of the present invention is to gain access to mascara compositions whose hue, notably in the range of the blacks, retains its intensity over time, notably over several days and according to temperature variations.

A second aim of the present invention is to gain access to mascara compositions whose texturing properties are finely adjustable and reproducible.

A third aim of the present invention is to gain access to mascara compositions for which the mechanical properties of the deposit of the product on keratin fibers, such as the resistance to flaking, are finely adjustable and reproducible.

Another aim of the present invention is to provide a mascara architecture produced using a predominant weight proportion of single-component ingredients. The predominant use of single-component ingredients advantageously makes it possible to eliminate the risk of composition variability that may exist between several production batches of a multicomponent ingredient and therefore to eliminate its impact on the final properties of the mascara.

Another aim of the present invention is to provide a mascara architecture which makes it possible to significantly reduce or even dispense with the use of waxes, but which nevertheless remains very satisfactory in terms of makeup effect.

In the course of their research, the inventors have discovered, unexpectedly, that the aims as defined previously are achieved by using a composition comprising, notably in a physiologically acceptable medium:
(1) at least one linear fatty acid monoester of formula (I) below:

[Chem 1]

R₁—O—R₂ (I)

in which R₁ and R₂ are linear and saturated and have, independently of each other, a number of carbon atoms greater than or equal to 20, with R₁ representing an acyl radical, and R₂ representing an alkyl radical, and
(2) at least one anionic surfactant chosen from:
   C₁₂-C₂₀ monoalkyl phosphates;
   fatty acids containing from 14 to less than 20 carbon atoms;
   mixtures thereof, and
(3) at least one base that is capable of partially or totally neutralizing said anionic surfactant (2),
(4) at least one vinylpyrrolidone/eicosene copolymer, and
(5) at least one dyestuff, and
(6) water, said composition comprising less than 5.0% by weight of wax(es) relative to the total weight of the composition, preferably less than 3% by weight of wax(es), and more particularly is free of wax.

Unexpectedly, the inventors have indeed found that the formulation comprising at least one linear fatty acid monoester (1), at least one anionic surfactant (2) in a form partially or totally neutralized with a base (3), at least one vinylpyrrolidone/eicosene copolymer (4), at least one dyestuff (5) and water (6), makes it possible to obtain compositions whose hue, more particularly in the range of the blacks, retains its intensity over time, notably over several days and according to temperature variations.

Furthermore, the texture and mechanical properties of the deposit of the formulation on the keratin fibers can be finely adjusted and guaranteed in terms of reproducibility.

As emerges from the text hereinbelow, these novel compositions are advantageous in several respects.

First of all, compounds (1) to (6) required according to the invention are as an individualized compound, a single-component compound or a compound with a well-defined number of components, as opposed to the majority of conventional waxes which are often multicomponent or even have an indefinite number of compounds, such as natural waxes and some synthetic waxes.

These two specificities are particularly advantageous since they make it possible to eliminate a risk of variability with regard to their respective compositions.

As emerges from the examples below, the compositions in accordance with the invention and based on the use of compounds (1) as texturing agent prove to be very satisfactory in terms of makeup effects.

Thus, compositions according to the invention may have a creamy texture which proves to be finely adjustable by virtue of the use of the required combination according to the invention.

The obtention of these properties is conditioned by the use of the compounds (1) to (6) and advantageously does not therefore require the additional presence of waxes, notably in significant amount.

Thus, the compositions according to the invention advantageously comprise less than 5.0% of waxes as defined below.

The term “waxes” refers to lipophilic compounds, which are solid at room temperature (25° C.) and at atmospheric pressure (760 mmHg), with a reversible solid/liquid change of state, which have a melting point of greater than or equal to 40° C., which may be up to 120° C.

For the purposes of the invention, the waxes to which this abovementioned amount limitation relates are distinct from those that may be embodied by the component which is a monoester of fatty acid(s) (1) required according to the invention and additives of the fatty alcohol type.

This discovery forms the basis of the invention.

### SUBJECTS OF THE INVENTION

Thus, according to one of its aspects, the present invention relates to a composition comprising, notably in a physiologically acceptable medium:
(1) at least one linear fatty acid monoester of formula (I) below:

[Chem 1]

R₁—O—R₂ (I)

in which R₁ and R₂ are linear and saturated and have, independently of each other, a number of carbon atoms greater than or equal to 20, with R1 representing an acyl radical, and R2 representing an alkyl radical, and
(2) at least one anionic surfactant chosen from:
   C₁₂-C₂₀ monoalkyl phosphates;
   fatty acids containing from 14 to less than 20 carbon atoms;
   mixtures thereof, and
(3) at least one base that is capable of partially or totally neutralizing said anionic surfactant,
(4) at least one vinylpyrrolidone/eicosene copolymer, and
(5) at least one dyestuff, and
(6) water.

A second subject of the present invention is a cosmetic process for making up and/or caring for human keratin materials, such as the skin, notably the area around the eyes, the contour of the eyelashes, the contour of the eyebrows; keratin fibers such as the eyelashes and the eyebrows, which consists in applying to said keratin materials a composition as defined previously.

### Definitions

In the context of the present invention, the term “keratin material” notably means the skin, notably the contour of the eyes, the contour of the eyelashes, the contour of the eyebrows; keratin fibers such as the eyelashes and the eyebrows. For the purposes of the present invention, this term “keratin fibers” also extends to synthetic false eyelashes.

The term “physiologically acceptable” means compatible with the skin and/or its integuments, which has a pleasant color, odor and feel, and which does not cause any unacceptable discomfort (stinging or tautness) liable to discourage the consumer from using this composition.

According to another of its aspects, the present invention relates to a process, notably a cosmetic process, for caring for and/or making up keratin materials, in particular the eyelashes and/or the eyebrows, comprising at least one step which consists in applying, to said keratin materials, in particular the eyelashes and/or the eyebrows,

### Fatty Acid Monoester

A composition according to the invention comprises at least one linear fatty acid monoester.

A composition according to the invention may comprise at least 5.0% by weight, preferably at least 6.0% by weight, better still at least 7.0% by weight of linear fatty acid monoester(s), relative to the total weight of the composition.

According to a particularly preferred embodiment of the invention, the fatty acid monoester(s) are present in the composition in a content ranging from 6.0% to 35.0% by weight, preferably from 7.0% to 30.0% in weight, or even preferably from 8.0% to 28.0% by weight, relative to the total weight of the composition.

The linear fatty acid monoester(s) (1) under consideration according to the invention correspond to formula (I) below:

R₁—O—R₂ (I)

in which R₁ and R₂ are linear and saturated and have, independently of each other, a number of carbon atoms greater than or equal to 20, with R₁ representing an acyl radical, and R₂ representing an alkyl radical.

This or these fatty acid monoester(s) are used during the preparation of a composition according to the invention, in an individualized form or in the form of a mixture comprising exclusively linear fatty acid monoesters of formula (I).

In a preferred embodiment, the fatty acid monoester(s) have a melting point greater than 50° C.

The melting point may be measured by any known method and in particular using a differential scanning calorimeter (DSC).

According to a preferred embodiment of the invention, the acyl and alkyl radicals representing respectively R₁ and R₂ are chosen in such a way that compound (I) is solid at a temperature of less than or equal to 30° C.

According to a particularly preferred embodiment of the invention, R₁ and R₂ are, respectively, acyl and alkyl radicals having a number of carbon atoms ranging from 20 to 30, preferably from 20 to 24.

According to a particularly preferred embodiment, R₁ and R₂ are, respectively, acyl and alkyl radicals having the same number of carbon atoms.

In particular, the fatty acid monoester according to the invention is chosen from arachidyl arachidate and behenyl behenate.

According to a particularly preferred embodiment of the invention, the linear fatty acid monoester is a behenyl behenate.

A behenyl behenate that is suitable for use in the composition according to the invention may notably be Kester Wax K-72® sold by the company Koster Keunen, Dub BB® sold by Stearinerie Dubois, or Dermowax BB® sold by Alzo.

### Anionic Surfactant

As specified above, the fatty acid monoester(s) (1) used according to the invention are combined with at least one anionic surfactant (2) chosen from:
C₁₂-C₂₀ monoalkyl phosphates;
fatty acids containing from 14 to less than 20 carbon atoms;
mixtures thereof, that is at least partially or totally neutralized with a base (3).

For the purposes of the present invention, the term “surfactant” means an amphiphilic chemical compound, that is to say a compound having in its structure two parts of different polarity. Generally, one is lipophilic (soluble or dispersible in an oily phase). The other is hydrophilic (soluble or dispersible in water). Surfactants are characterized by the value of their HLB (hydrophilic-lipophilic balance), the HLB being the ratio of the hydrophilic part to the lipophilic part in the molecule. The term “HLB” is well known to those skilled in the art and is described, for example, in “The HLB System. A Time-Saving Guide to Emulsifier Selection” (published by ICI Americas Inc.; 1984).

According to a preferential form of the invention, the anionic surfactants in accordance with the invention have an HLB of greater than or equal to 8. The HLB of the anionic surfactant(s) used according to the invention may be determined by the Griffin method.

The term “anionic surfactant” means any negatively charged amphiphilic molecule.

According to a preferred embodiment of the invention, the anionic surfactant is present in a content ranging from 3.5% to 20.0% by weight, more preferentially from 4.0% to 20.0% by weight, better still from 4.5% to 15.0% by weight, even better still from 5.0% to 15.0% by weight relative to the total weight of the composition.

a) C₁₂-C₂₀ Monoalkyl Phosphates

The designation “monoalkyl” means the phosphate element is linked to a single C₁₂-C₂₀ alkyl chain, unless otherwise specified. The monoalkyl phosphate(s) (including phosphine oxide(s)) that may be used in the compositions according to the present application is/are chosen from C₁₄-C₂₀ and preferably C₁₆-C₁₈ monoalkyl phosphates and mixtures thereof.

Preferably, they are chosen from monocetyl phosphate, monostearyl phosphate and monocetearyl phosphate.

In particular, use will be made of the anionic surfactant in its form totally neutralized with potassium hydroxide, namely the potassium salt of monocetyl phosphate having the INCI name Potassium Cetyl Phosphate, for example sold under the names Amphisol K® (DSM Nutritional Products), Amphisol A® (DSM Nutritional Products), Arlatone MAP® (Uniqema), Crodafos MCA® (Croda).

b) Fatty Acid Containing from 14 to Less than 20 Carbon Atoms

The fatty acid according to the invention comprises from 14 to less than 20 carbon atoms. According to a preferred embodiment of the invention, the fatty acid comprises from 16 to less than 20 carbon atoms. According to a particularly preferred embodiment, the number of carbon atoms ranges from 16 to 18.

In particular, the fatty acid(s) according to the invention are chosen from linear fatty acids, saturated fatty acids and mixtures thereof.

According to a particularly advantageous embodiment of the invention, the fatty acid of the ionic surfactant is linear and saturated.

According to a particular embodiment of the invention, the fatty acid(s) are chosen from palmitic acid, stearic acid and mixtures thereof, and preferably comprise at least the stearic acid having the INCI name Stearic Acid.

Thus, according to another embodiment of the invention, the composition uses, as fatty acid (2), a mixture of C₁₆-C₁₈ fatty acids, preferably a mixture of fatty acids containing 16 carbon atoms, such as palmitic acid, and of fatty acids containing 18 carbon atoms, such as stearic acid.

A preferred stearic acid that is suitable for use in the invention is, for example, Stearic Acid 1850® sold by the company Southern Acids or the product Stearine TP 1200 Pastilles® (DUB 50P®) by the company Stearinerie Dubois.

### Base

The composition according to the invention comprises at least one base. This base may be organic or inorganic.

According to a first variant, the base is at least one organic base.

Preferably, the base of organic origin is chosen from amino acids such as arginine; alkanolamines such as monoethanolamine, diethanolamine, triethanolamine, triisopropanolamine, aminomethylpropanol; primary (poly)hydroxyalkylamines such as 2-amino-2-(hydroxymethyl) propane-1,3-diol (also known as tromethamine) and aminomethylpropanediol; and mixtures thereof.

According to a particular embodiment, the base is a primary (poly)hydroxyalkylamine.

According to another particular embodiment, the base is an amino acid, in particular arginine.

The term “primary (poly)hydroxyalkylamine” in particular means a primary dihydroxyalkylamine, it being understood that the term “primary” means a primary amine function, i.e. —NH₂, and the alkyl group being a linear or branched C₁-C₈ and preferably a branched C₄ hydrocarbon-based chain, such as 1,3-dihydroxy-2-methylpropyl. The primary (poly)hydroxyalkylamine is preferentially 1,3-dihydroxy-2-methyl-2-propylamine (also known as aminomethylpropanediol or AMPD).

According to a preferred embodiment of the invention, the base of organic origin is aminomethylpropanediol.

Such an aminomethylpropanediol that is suitable for use in the invention is, for example, AMPD Ultra PCR sold by the company Angus (Dow Corning).

According to a second variant, the base is at least one inorganic base.

This inorganic base is chosen from ammonium (NH₄⁺) and alkali metal hydroxides.

Preferably, the inorganic base is chosen from sodium hydroxide and potassium hydroxide, and mixtures thereof.

The amount of base is adjusted so as to obtain sufficient neutralization to confer effective ionicity to the associated fatty acid (2).

Preferably, the base is present in an amount that is sufficient to neutralize some or all of the carboxylic functions of the fatty acid(s) (2) comprising from 14 to less than 20 carbon atoms.

For example, the composition according to the invention may comprise at least 0.1% by weight, better still at least 0.15% by weight, of base(s), relative to the total weight of the composition.

According to a preferred embodiment of the invention, the base is present in a content ranging from 0.2% to 3.0% by weight, preferably from 0.3% to 2.0% by weight of base(s), in particular of sodium hydroxide, potassium hydroxide or aminomethylpropanediol, relative to the total weight of the composition according to the invention.

According to a particular mode of the invention, the compositions according to the invention contain stearic acid neutralized with sodium hydroxide.

According to another particular mode of the invention, the compositions according to the invention contain stearic acid neutralized with aminomethylpropanediol.

The anionic surfactant (2) and the base (3) making up the neutralized ionic surfactant according to the invention may be introduced into the composition in the form of one and the same commercial material, or one after the other in the form of two distinct commercial materials.

Preferably, the fatty acid (2) and the base (3) will be introduced into the composition in the form of two distinct commercial materials.

Preferably, the C₁₂-C₂₀ monoalkyl phosphate (2) and the base (3) are introduced into the composition as one and the same commercial material.

### Vinylpyrrolidone/Eicosene Copolymer

The composition according to the invention comprises at least one copolymer of vinylpyrrolidone and eicosene (C₂₀ olefin) of the following structure:

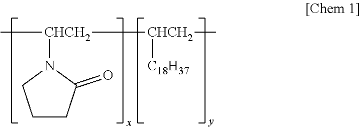

and having the INCI name: VP/Eicosene Copolymer, such as the commercial products sold under the names Antaron V-220® and Ganex V-220® by the company Ashland Inc. and Unimer U-15® by the company Givaudan Active Beauty.

The composition according to the invention comprises at least one copolymer of vinylpyrrolidone and eicosene, preferably in a concentration ranging from 0.5% to 10% by weight relative to the total weight of the composition, preferably ranging from 1% to 5% by weight relative to the total weight of the composition.

### Dyestuff

A composition according to the invention, and notably those intended for makeup, generally comprises at least one dyestuff such as pulverulent dyestuffs, liposoluble dyes or water-soluble dyes.

The pulverulent dyestuffs may be chosen from pigments and nacres.

The pigments may be white or colored, mineral and/or organic, and coated or uncoated. Among the mineral pigments that may be mentioned are titanium dioxide, optionally surface-treated, zirconium oxide, zinc oxide or cerium oxide, and also iron oxide, chromium oxide, manganese violet, ultramarine blue, chromium hydrate and ferric blue. Among the organic pigments that may be mentioned are carbon black, pigments of D & C type and lakes based on cochineal carmine or on barium, strontium, calcium or aluminum.

The nacres may be chosen from white nacreous pigments such as mica coated with titanium or with bismuth oxychloride, colored nacreous pigments such as titanium mica with iron oxides, titanium mica notably with ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, B-carotene, soybean oil, Sudan Brown, D&C Yellow 11, D&C Violet 2, D&C Orange 5, quinoline yellow and annatto.

Preferably, the composition according to the invention comprises a pulverulent dyestuff, preferably of pigment type, in particular metal oxides, notably titanium dioxides, iron oxides and mixtures thereof; and more preferentially iron oxides, and more particularly black iron oxides (CI 77499).

Preferably, said dyestuff is present in the composition in a content ranging from 2.0% to 25.0% by weight, preferably from 3.0% to 20.0% by weight, more particularly from 4.0% to 15.0% by weight, relative to the total weight of the composition.

### Water

A composition according to the invention comprises water.

A water suitable for the invention may be a floral water such as cornflower water and/or a mineral water such as Vittel water, Lucas water or La Roche Posay water and/or a spring water.

In particular, a composition according to the invention comprises at least 30.0% by weight, better still at least 40.0% by weight, or even a content of from 50% to 60% by weight of water, relative to the total weight of the composition.

In a preferred embodiment of the invention, the composition according to the invention includes:
(1) at least behenyl behenate, and
(2) at least stearic acid neutralized with (3) aminomethylpropanediol, and
(4) at least one black iron oxide (CI 77499), and
(5) water.

### Other Components

In addition to the abovementioned compounds, a composition according to the invention may, needless to say, comprise additional ingredients.

### a) Fatty Alcohol

Preferentially, the compositions according to the invention also comprise at least one fatty alcohol. A composition can therefore comprise a single fatty alcohol according to the invention or several distinct fatty alcohols.

If several distinct fatty alcohols are present, they can be added separately during the preparation of the composition and the mixture thereof can then be formed in situ. They can also be used in the form of a mixture which is already commercially available and in which the weight proportion and the degree of purity of each of the fatty alcohols are controlled. In other words, the composition of these mixtures is faithfully reproducible, as opposed to mixtures of fatty alcohols generated via synthesis from starting materials derived from complex mixtures.

The fatty alcohol(s) are in particular chosen from linear or branched, saturated or unsaturated C₁₄-C₃₀, preferably C₁₄-C₂₄, and better still C₁₄-C₂₀, fatty alcohols.

The fatty alcohol(s) are in particular chosen from linear and saturated C₁₄-C₃₀ fatty alcohols, preferably linear and saturated C₁₄-C₂₄ fatty alcohols, and better still linear and saturated C₁₄-C₂₀ fatty alcohols.

According to a particularly preferred embodiment, the fatty alcohol is in the form of a mixture of several different fatty alcohols, and is preferably a mixture of several linear and saturated C₁₄-C₃₀, better still C₁₄-C₂₄, even better still C₁₄-C₂₀, fatty alcohols.

Preferably, the fatty alcohol according to the invention is chosen from cetyl alcohol (C₁₆), stearyl alcohol (C₁₈) and mixtures thereof (also known as “cetearyl alcohol”). Preferentially, the fatty alcohol according to the invention is a mixture of cetyl alcohol and stearyl alcohol. Such a mixture is notably sold under the name Lannette O OR/MB® by the company BASF.

According to a preferred embodiment, the fatty alcohol is solid at room temperature.

The fatty alcohol is present in the compositions of the invention in amounts ranging from 1.0% to 20.0% by weight, relative to the total weight of the composition, preferably from 2.0% to 15.0% by weight and even more particularly from 3.0% to 10.0% by weight, relative to the total weight of the composition.

### b) Waxes

Thus, a composition according to the invention may also comprise a wax.

However, with regard to the objectives targeted by the present invention, the compositions according to the invention comprise a reduced amount of wax(es) of less than 5.0% by weight, or even less than 3.0% by weight of waxes, relative to the total weight thereof.

As specified in the preamble, for the purposes of the invention, the term “waxes” refers to lipophilic compounds, which are solid at room temperature (25° C.) and at atmospheric pressure (760 mmHg), with a reversible solid/liquid change of state, which have a melting point of greater than or equal to 40° C., which may be up to 120° C.

It is recalled that, for the purposes of the invention, the waxes to which the abovementioned amount limitation relates are distinct from those that may be embodied by the component which is a linear fatty acid monoester (1) according to the invention and additional fatty alcohol components as defined below.

This limitation relates more particularly to waxes made up of complex mixtures and which are notably described in Ullmann's Encyclopedia of Industrial Chemistry 2015, Wiley-VCH Verlag Gmbh & Co. KGaA.

Such waxes may notably be natural, but may also be synthetic.

The term “natural” wax is intended to denote any wax which pre-exists in nature or which can be converted, extracted or purified from natural compounds which exist in nature.

Among natural waxes, mention may notably be made of waxes termed fossil waxes, including those of petroleum origin, such as ozokerite, pyropissite, macrocrystalline waxes, also known as paraffins—including crude or gatsch waxes, gatsch raffinates, de-oiled gatsch, soft waxes, semirefined waxes, filtered waxes, refined waxes—and microcrystalline waxes, termed microwaxes, including bright stock gatsch. The fossil waxes also contain lignite, also known as montan wax, or peat wax.

As natural waxes other than fossil waxes, mention may be made of animal and plant waxes.

As examples of plant waxes, mention may be made of carnauba wax, candelilla wax, ouricury wax, sugarcane wax, jojoba wax, Trithrinax campestris wax, raffia wax, alfalfa wax, wax extracted from Douglas fir, sisal wax, flax wax, cotton wax, Batavia dammar wax, cereal wax, tea wax, coffee wax, rice wax, palm wax, Japan wax, mixtures thereof and derivatives thereof.

As examples of animal waxes, mention may be made of beeswax, Ghedda wax, shellac, Chinese wax, lanolin, also known as wool wax, mixtures thereof and derivatives thereof.

These waxes are generally multicomponent. For example, natural beeswax is approximately 70% composed of esters which are mainly monoesters (of fatty acid and of fatty alcohol), but also of hydroxy esters, of diesters and triesters and esters of sterols, and also of long-chain linear hydrocarbons, of free acids and of free alcohols. For obvious reasons, the weight proportion of their ingredients and their degree of purity are difficult to guarantee from one production batch to another.

The term “synthetic” wax is intended to denote waxes whose synthesis requires one or more chemical reactions performed by a human being.

Among the synthetic waxes, semisynthetic waxes and totally synthetic waxes can be distinguished. Synthetic waxes may be waxes obtained via a Fischer-Tropsch process, constituted for example of paraffins with a number of carbon atoms ranging from 20 to 50 or waxes of polyolefins, for example homopolymers or copolymers of ethylene, of propene or butene, or even longer-chain α-olefins. The latter can be obtained by thermomechanical degradation of polyethylene plastic, by the Ziegler process, by high-pressure processes, or else via processes catalyzed by metallocene species. These waxes may be crystallizable, partially crystallizable or amorphous. The abovementioned synthetic waxes are generally apolar and can be chemically treated to obtain polar waxes, for example via one or more of the following reactions: air oxidation, grafting, esterification, neutralization with metal soaps, amidation, direct copolymerizations or addition reactions.

In this case also, their composition may be constituted of a mixture of ingredients since the fatty-chain lengths are not well defined, thus forming a mixture of compounds having different fatty-chain lengths and for which it is difficult for manufacturers to guarantee perfect reproducibility from one production batch to another.

Consequently, the compositions according to the invention advantageously comprise less than 5% by weight, preferably less than 3% by weight of waxes, notably of multicomponent natural or synthetic wax, relative to the total weight of the composition.

For the purposes of the invention, a multicomponent wax denotes a wax consisting of a mixture of several ingredients, either such as exists naturally like natural waxes, or such as is formed during the process of industrial synthesis of these materials.

In a particularly preferred embodiment of the invention, the composition is free of these waxes, notably multicomponent natural or synthetic wax.

As specified above, the preferred texturing compounds according to the invention are by contrast and advantageously synthetic, single-component compounds, which are thus available in a form purified to more than 99%, like compound (1) required according to the invention.

### c) Liquid Fatty Phase

A composition according to the invention may also comprise a liquid fatty phase.

Such a liquid fatty phase is an organic phase that is liquid at room temperature (25° C.) and at atmospheric pressure (760 mmHg), nonaqueous and water-immiscible.

The liquid fatty phase may contain a nonvolatile oil notably chosen from nonvolatile hydrocarbon-based oils.

The term “hydrocarbon-based oil” refers to an oil mainly containing carbon and hydrogen atoms and possibly one or more functions chosen from hydroxyl, ester, ether and carboxylic functions.

The term “nonvolatile oil” refers to an oil that remains on the skin or the keratin fiber at room temperature and atmospheric pressure for at least several hours, and that notably has a vapor pressure of less than 2.66 Pa, preferably less than 0.13 Pa. By way of example, the vapor pressure may be measured via the static method or via the effusion method by isothermal thermogravimetry, depending on the vapor pressure (standard OCDE 104).

A composition according to the invention may comprise from 1.0% to 20.0% by weight, from 2.0% to 12.0% by weight, and preferentially from 2.0% to 8.0% by weight of volatile oil, relative to the total weight of the composition. [00131] A composition according to the invention preferably comprises less than 5.0% by weight and more preferentially less than 2.0% by weight of volatile oil(s), relative to the total weight of the composition. In one particularly preferred embodiment of the invention, the composition is free of volatile oils.

The term “volatile oil” refers to an oil that can evaporate on contact with the skin in less than one hour, at room temperature (25° C.) and atmospheric pressure (760 mmHg). More specifically, a volatile oil has an evaporation rate ranging from 0.01 to 200 mg/cm²/min.

### d) Water-Soluble Solvents

A composition according to the invention may also additionally comprise at least one water-soluble solvent.

In the present invention, the term “water-soluble solvent” denotes a compound that is liquid at room temperature and water-miscible (miscibility with water of greater than 50% by weight at 25° C. and atmospheric pressure).

The water-soluble solvents that may be used in the composition of the invention may also be volatile.

The water-soluble solvents that may be used in the composition of the invention may also be volatile.

Among the water-soluble solvents that may be used in the composition in accordance with the invention, mention may be made notably of lower monoalcohols containing from 1 to 5 carbon atoms such as ethanol and isopropanol, glycols containing from 2 to 8 carbon atoms such as ethylene glycol, propylene glycol, 1,3-butylene glycol, propanediol, pentylene glycol, glycerol and dipropylene glycol, C₃-C₄ ketones and C₂-C₄ aldehydes.

### e) Hydrophilic Film-Forming Polymer(s)

A composition according to the invention may preferably comprise at least one hydrophilic film-forming polymer.

For the purposes of the present invention, the term “hydrophilic polymer” means a water-soluble polymer.

For the purposes of the present invention, the term “water-soluble polymer” refers to a polymer which, when introduced into water at a concentration equal to 1%, gives a macroscopically homogeneous solution whose light transmittance, at a wavelength equal to 500 nm, through a sample 1 cm thick, is at least 10%.

For the purposes of the present invention, the term “film-forming polymer” refers to a polymer that is capable, by itself or in the presence of an auxiliary film-forming agent, of forming a macroscopically continuous deposit, and preferably a cohesive deposit, and even better still a deposit whose cohesion and mechanical properties are such that said deposit is isolable and can be manipulated individually, for example when said deposit is prepared by pouring onto a nonstick surface such as a Teflon-coated or silicone-coated surface.

For the purposes of the invention, a hydrophilic film-forming polymer that is particularly advantageous is a (poly)vinylpyrrolidone hydrophilic polymer.

A (poly)vinylpyrrolidone hydrophilic polymer that is suitable for use in the invention may have a weight-average molecular mass, Mw, ranging from 1500 to 500 000 g/mol.

A composition according to the invention has a total solids content of (poly)vinylpyrrolidone hydrophilic polymer(s) of greater than or equal to 0.5% by weight, preferably greater than or equal to 1.0% by weight, more preferentially greater than or equal to 1.5% by weight, relative to the total weight of the composition.

The composition according to the invention preferably comprises from 0.1% to 15.0% by weight, preferably from 0.5% to 10.0% by weight, better still from 1.0% to 8.0% by weight of hydrophilic film-forming polymer(s), relative to the total weight of the composition.

Preferably, the (poly)vinylpyrrolidone hydrophilic polymer(s) in accordance with the invention are linear.

In particular, the (poly)vinylpyrrolidone hydrophilic polymer(s) in accordance with the invention are chosen from random polymers, block copolymers, and a mixture thereof. The term “block copolymer” means a polymer comprising at least two different blocks or sequences and preferably at least three different blocks.

The (poly)vinylpyrrolidone hydrophilic polymer(s) are chosen from:
(poly)vinylpyrrolidone homopolymers;
copolymers of (poly)vinylpyrrolidone/(poly)vinyl acetate, esters,
copolymers of (poly)vinylpyrrolidone/(meth)acrylic, salts thereof, thereof and a mixture thereof.

As (poly)vinylpyrrolidone homopolymers, examples that may be mentioned include:
the polyvinylpyrrolidone (2500 g/mol) sold under the trade name Kollidon 17 PF by the company BASF,
the polyvinylpyrrolidone sold under the trade name Luviskol K 30 Powder by the company BASF or sold under the trade name PVP K 30L® by the company ISP (Ashland),
the polyvinylpyrrolidone sold under the trade name PVP K 90® by the company ISP (Ashland).

As (poly)vinylpyrrolidone/(poly)vinyl acetate copolymers, examples that may be mentioned include the vinylpyrrolidone/vinyl acetate (60/40) copolymer sold under the trade name Luviskol VA 64 Powder by the company BASF.

### f) Cosmetic Active Agents

As cosmetic active agents that may be used in the compositions according to the invention, mention may be made notably of antioxidants, preserving agents, fragrances, neutralizers, cosmetic active agents, for instance emollients, vitamins and screening agents, in particular sunscreens, and mixtures thereof.

These additives may be present in the composition in a content ranging from 0.01% to 15.0% relative to the total weight of the composition.

Needless to say, a person skilled in the art will take care to select the optional additional additives and/or the amount thereof such that the advantageous properties of the composition according to the invention are not, or are not substantially, adversely affected by the envisaged addition.

### Physical Characteristics

### a) Solids Content

The composition according to the invention advantageously has a solids content of at least 25.0% by weight, and preferentially of at least 30.0% by weight, relative to the total weight of the composition, or even from 30.0% to 60.0% by weight, relative to the total weight of the composition.

For the purposes of the present invention, the term “solids content” denotes the content of nonvolatile matter.

The amount of solids content (abbreviated as SC) of a composition according to the invention is measured using a Halogen Moisture Analyzer HR 73® commercial halogen desiccator from Mettler Toledo. The measurement is performed on the basis of the weight loss of a sample dried by halogen heating, and thus represents the percentage of residual matter once the water and the volatile matter have evaporated off.

This technique is fully described in the machine documentation supplied by Mettler Toledo®.

The measuring protocol is as follows:

Approximately 2 g of the composition, referred to hereinbelow as the sample, are spread out on a metal crucible, which is placed in the halogen desiccator mentioned above. The sample is then subjected to a temperature of 105° C. until a constant weight is obtained. The wet mass of the sample, corresponding to its initial mass, and the dry mass of the sample, corresponding to its mass after halogen heating, are measured using a precision balance.

The experimental error associated with the measurement is of the order of plus or minus 2%.

The solids content is calculated in the following manner:

$$[ Math 1 ]$$

$$Solids content ( expressed in \% by weight ) = 10 \; 0 \times \frac{Dry mass}{Wet mass}$$

### b) Consistency

Preferably, a composition according to the invention has a consistency at 25° C. characterized by a hardness≥20 g, more preferentially a hardness ranging from 20 to 200 g, even more preferentially from 30 to 150 g.

The consistency of the compositions of the invention is measured according to the following protocol:

The measuring device is a TA-XT-Plus® sold by the company Staples Micro System, equipped with a cell for measuring a force of 5 kg and with a cylindrical spindle 12.7 mm (½ inch) in diameter made of Delrin. The mascara is thermostatically maintained at 20° C. It is placed in excess in a container 60 mm in diameter and 22 mm deep using a metal spatula. The product is spread out so as to avoid any air pockets but without triturating it, so as not to destructure it. The container is then leveled off using a spatula so as to have a surface that is as uniform as possible. The container is then covered with a watch glass so as to limit the evaporation of solvents present in the formula for about 10 minutes. The options chosen for this measuring method are the following:
Test mode: Compression measurement
Trigger force: 2.0 g
Pre-speed: 0.5 mm/sec
Test speed: 0.5 mm/sec
Temperature: 20° C.±1° C.
Penetration distance: 5 mm

Three successive measurements are taken at points at least 12 mm apart, at least 10 mm from the edge of the container. The container is held in place during the measurement. The value retained is the mean of the maxima obtained on each measurement.

Such a consistency is particularly advantageous since it is the most suitable for the mascara application device.

c) Color Intensity Stability of the Composition with Respect to Temperature Variations

The composition is monitored under extreme temperature conditions. The mascara is placed in a hermetically sealed 100 ml glass jar and then placed in a Leasametric® brand Vötsch VT3050® temperature cycle oven ranging from 4 to 45° C. for 7 days according to the following program:
1 hour at 20° C.
then 11 hours at 4° C.
and finally 12 hours at 45° C.

The change from 4 to 45° C. takes place over the course of about 20 to 30 min. The intensity of the color

The color of the composition is evaluated by colorimetry in the CIE L*a*b* system using a Konica®/Minolta CM 600D® spectrocolorimeter with CM-100W Spectra Magic NX® software (illuminant D65, specular components excluded, 8 mm aperture). In this L*a*b* system, L* represents the color intensity, a* indicates the green/red color axis and b* the blue/yellow color axis.

The stability of the color intensity after 7 days of storage in the cycle oven was measured by

ΔL*=(L*−L*₀)
where
L*₀ is the mean intensity of the color on six measurements, measured between 24 hours and 72 hours after the day of manufacture of the composition, and
L* is the mean intensity of the color of the mascara after 7 days of storage in the cycle oven and L*₀ is the mean intensity of the color of the mascara measured at the time when the pill box is introduced at the start of the test. The lower the value of ΔL*, the greater the stability of the color.

The composition may be manufactured via the known processes generally used in the cosmetics field.

The composition used according to the invention may be a makeup composition, a makeup base, notably for keratin fibers, or base coat, a composition to be applied onto makeup, also known as topcoat, or else a composition for treating keratin fibers.

More specifically, the composition according to the invention is in the form of an eyelash product such as a mascara, an eyebrow product or an eye contour product such as an eye liner, and more particularly a mascara.

Such compositions are notably prepared according to the general knowledge of a person skilled in the art.

### Packaging and Application Assembly or Kit

The present invention also relates to an assembly, or kit, for packaging and applying a cosmetic composition for coating keratin fibers, comprising:
a packaging device comprising the cosmetic composition for coating keratin fibers as described previously,
an applicator for said composition.

Said applicator may be integrally attached to a handle member forming a cap for said packaging device. In other words, said applicator may be mounted in a removable position on said device between a closed position and an open position of a dispensing aperture of the device for packaging said composition.

An assembly for coating keratin fibers that is suitable for use according to the invention may comprise an applicator suitable for applying said cosmetic composition for coating keratin fibers and, where appropriate, a conditioning device suitable for receiving said composition.

### Applicator

The applicator comprises means for smoothing and/or separating keratin fibers, such as the eyelashes or the eyebrows, notably in the form of teeth, bristles, spikes or other reliefs.

The applicator is arranged to apply the composition onto the eyelashes or the eyebrows, and may include, for example, a brush or a comb.

The applicator may also be used for finishing of the makeup, over a region of the eyelashes or eyebrows that is made up or laden with composition.

The brush may comprise a twisted core and bristles held between the turns of the core, or may be made in yet another way.

The comb is, for example, produced from a single part by molding of a plastic.

In certain exemplary embodiments, the application member is mounted at the end of a wand, which wand may be flexible, which may contribute to improving the comfort during application.

### Packaging Device

The packaging device comprises a container for housing the composition for coating keratin fibers. This composition may then be withdrawn from the container by immersing the applicator therein.

This applicator may be firmly attached to a member for closing the container. This closing member may form a member for handling the applicator. This handle member may form a cap to be removably mounted on said container by any suitable means, such as by screwing, click-fastening, coupling, etc. Such a container may thus reversibly house said applicator.

This container can be optionally equipped with a wiper suitable for removing a surplus of product taken up by the applicator.

A process for applying the composition according to the invention to the eyelashes or the eyebrows may also include the following steps:
forming a deposit of the cosmetic composition on the eyelashes or the eyebrows,
leaving the deposit on the eyelashes or the eyebrows, it being possible for the deposit to dry.

It should be noted that, according to another embodiment, the applicator may form a product container. In such a case, a container may, for example, be provided in the handle member and an internal channel can internally connect this handle member to the application members in relief.

Finally, it should be noted that the packaging and application assembly may be in the form of a kit, it being possible for the applicator and the packaging device to be housed separately in the same packaging article.

The expressions “between . . . and . . . ”, and “ranging from . . . to . . . ” should be understood as meaning limits included, unless otherwise specified.

In the description and the examples, the percentages are weight percentages, unless otherwise indicated. The percentages are thus expressed by weight, with respect to the total weight of the composition. The ingredients are mixed in the order and under the conditions that are readily determined by a person skilled in the art.

The invention will now be described by means of examples which are presented for purely illustrative purposes and should not be interpreted as examples that limit the invention.

### Examples 1, 1a and 1b of Mascara

The following Examples 1, 1a and 1b of mascara were made.

TABLES 1

| | Ex. 1 | Ex. 1a | Ex. 1b |
|---|---|---|---|
| | according | outside | outside |
| | to the | the | the |
| Ingredients (INCI name) | invention | invention | invention |
| Cetearyl alcohol | 5.0 | 5.0 | 5.0 |
| (Lanette O OR ®) | | | |
| Behenyl behenate (C44) | 8.5 | 8.5 | 8.5 |
| (fatty acid monoester (1)) | | | |
| (Kester Wax K-72 ®) | | | |
| VP/eicosene copolymer (4) | 5.0 | | — |
| (Antaron V 220F ®) | | | |
| Hydrogenated coco-glyceride | — | 5.0 | |
| (Softisan 100 ®) | | | |
| Triacontanyl PVP | | — | 5.0 |
| (Antaron WP 660 ®) | | | |
| Stearic acid | 6.0 | 6.0 | 6.0 |
| (fatty acid (2)) | | | |
| (Stearine TP 1200 Pastilles) | | | |
| (Dub 50P ®)) | | | |
| Iron oxides/CI 77499 | 10.0 | 10.0 | 10.0 |
| (dyestuff (5)) | | | |
| (Sunpuro Black Iron Oxide | | | |
| C33-7001 ® from Sun) | | | |
| PVP | 2.0 | 2.0 | 2.0 |
| (PVP K 30L ®) | | | |
| Phenoxyethanol | 0.6 | 0.6 | 0.6 |
| Capryl glycol | 0.6 | 0.6 | 0.6 |
| Aminomethyl propanediol | 1.0 | 1.0 | 1.0 |
| (base (3)) | | | |
| (AMPD Ultra PC ®) | | | |
| Water (6) | qs 100 | qs 100 | qs 100 |

### Protocol for Preparing Examples 1, 1a and 1b

The compositions were prepared on a Maxilab Olsa® jacketed tank as follows (on 2 kg):

At room temperature (25° C.), all the ingredients were introduced into the tank. The mixture was heated to about 90° C. with stirring. Emulsification was performed for 15 minutes under turbine/blade/scraper stirring (speed 3000/30/42 rpm)

The juice was gradually cooled to 60° C. under paddle/scraper stirring (speed=30/42 rpm) at a rate of 2° C./min, then to room temperature (25° C.) under paddle/scraper stirring (speed=30/42 rpm) at a rate of 2° C./min and deaerated before 35° C. under vacuum.

### Packaging of the Formulation

Each mascara formula obtained was then transferred to a glass jar hermetically sealed to avoid drying-out of the formula on contact with air.

### Comparative Tests of Intensity Stability of the Color Black

The stability of the intensity of the black color of Examples 1, 1a and 1b was evaluated after 7 days of storage in a Vötsch® VT3050 temperature cycle oven from the company Leasametric, according to the method indicated previously. The following are measured for each of the samples:
L*₀, the mean intensity of the black color on six measurements, measured between 24 hours and 72 hours after the day of manufacture of the composition to be evaluated.
L* the mean intensity of the black color on six measurements, after 7 days of storage in the temperature cycle oven.

The stability of the intensity of the black color after 7 days of storage in the temperature cycle oven was measured by

$$Δ {L}^{⋆} = ( {L}^{⋆} - {L}_{0}^{⋆} )$$

The results obtained are shown in the table below.

TABLES 2

| | Ex. 1 | Ex. 1a | Ex. 1b |
|---|---|---|---|
| | according to | outside the | outside the |
| Tests performed | the invention | invention | invention |
| ΔL* = (L* − L*o) after 7 days | 0.21 | −1.12 | 0.77 |
| in the cycle oven | | | |

It was observed that Example 1 according to the invention, comprising VP/eicosene copolymer, after 7 days in the cycle oven, has a black color whose intensity is more stable than that of Example 1a outside the invention, the composition of which is identical but which instead comprises Hydrogenated cocoyl glyceride, and more stable than that of Example 1b outside the invention, the composition of which is identical but which instead comprises Triacontanyl PVP.

1. A composition comprising, notably in a physiologically acceptable medium:
(1) at least one linear fatty acid monoester of formula (I) below:
[Chem 1]
R₁—O—R₂ (I)
in which R₁ and R₂ are linear and saturated and have, independently of each other, a number of carbon atoms greater than or equal to 20, with R₁ representing an acyl radical, and R₂ representing an alkyl radical, and
(2) at least one anionic surfactant chosen from:
C₁₂-C₂₀ monoalkyl phosphates;
fatty acids containing from 14 to less than 20 carbon atoms;
mixtures thereof, and
(3) at least one base that is capable of partially or totally neutralizing said anionic surfactant (2),
(4) at least one vinylpyrrolidone/eicosene copolymer, and
(5) at least one dyestuff, and
(6) water, said composition comprising less than 5.0% by weight of wax(es) relative to the total weight of the composition, preferably less than 3% by weight of wax(es), and more particularly is free of wax.

2. The composition as claimed in claim 1, in which the linear fatty acid monoester (1) is chosen from arachidyl arachidate and behenyl behenate, and preferably the linear fatty acid monoester is behenyl behenate.

3. The composition as claimed in any one of the preceding claims, comprising at least 5.0% by weight, preferably at least 6.0% by weight, better still at least 7.0% by weight of linear fatty acid monoester(s) (1) relative to the total weight of the composition; in particular, the content of linear fatty acid monoester(s) ranges from 6.0% to 35.0% by weight, preferably from 7.0% to 30.0% by weight, more particularly from 8.0% to 28.0% by weight, relative to the total weight of the composition.

4. The composition as claimed in any one of the preceding claims, in which said anionic surfactant is present in a content ranging from 3.5% to 20.0% by weight, more preferentially from 4.0% to 20.0% by weight, better still from 4.5% to 15.0% by weight, even better still from 5.0% to 15.0% by weight, relative to the total weight of the composition.

5. The composition as claimed in any of the preceding claims, in which said anionic surfactant(s) are chosen from fatty acids containing from 14 to less than 20 carbon atoms.

6. The composition as claimed in claim 5, in which the fatty acid(s) are chosen from stearic acid, palmitic acid and cetylic acid, and mixtures thereof, and is preferably stearic acid.

7. The composition as claimed in any one of the preceding claims, in which the base is at least one inorganic base, in particular chosen from sodium hydroxide, potassium hydroxide and ammonium, and mixtures thereof, and is preferably sodium hydroxide.

8. The composition as claimed in any one of the preceding claims, in which the base is at least one organic base, preferably amino acids, amines, primary (poly)hydroxyalkylamines, and mixtures thereof; in particular chosen from arginine, triethanolamine, aminomethylpropanol, aminomethylpropanediol, and mixtures thereof; and more particularly aminomethylpropanediol.

9. The composition as claimed in any one of the preceding claims, comprising at least 0.1% by weight, better still at least 0.15% by weight of base(s) (3), relative to the total weight of the composition; better still, the content of base(s) ranges from 0.2% to 3.0% by weight, preferably from 0.3% to 2.0% by weight, relative to the total weight of the composition.

10. The composition as claimed in any one of the preceding claims, comprising at least one copolymer of vinylpyrrolidone and eicosene, in a concentration ranging from 0.5% to 10% by weight relative to the total weight of the composition, preferably ranging from 1% to 5% by weight relative to the total weight of the composition.

11. The composition as claimed in any one of the preceding claims, comprising at least 30.0% by weight, better still at least 40.0% by weight, or even a content of from 50% to 60% by weight of water, relative to the total weight of the composition.

12. The composition as claimed in any one of the preceding claims, in which the dyestuff(s) are present in the composition in a content ranging from 2.0% to 25.0% by weight, preferably from 3.0% to 20.0% by weight, more particularly from 4.0% to 15.0% by weight, relative to the total weight of the composition.

13. The composition as claimed in any one of the preceding claims, in which the dyestuff is chosen from pulverulent dyestuffs, liposoluble dyes and water-soluble dyes; preferentially chosen from metal oxides; more particularly chosen from iron oxides, and more particularly black iron oxides (CI 77499).

14. The composition as claimed in any one of the preceding claims, also comprising at least one fatty alcohol, preferably chosen from linear and saturated C₁₄-C₃₀ fatty alcohols; better still chosen from linear and saturated C₁₄-C₂₄ fatty alcohols; more particularly chosen from linear and saturated C₁₄-C₂₀ fatty alcohols; even better still chosen from cetyl alcohol and stearyl alcohol, and mixtures thereof.

15. The composition as claimed in claim 14, in which the fatty alcohol is present in amounts ranging from 1.0% to 20.0% by weight, relative to the total weight of the composition, preferably from 2.0% to 15.0% by weight, and even more particularly from 3.0% to 10.0% by weight, relative to the total weight of the composition.

16. The composition as claimed in any one of the preceding claims, also comprising at least one water-soluble film-forming polymer, preferably a vinylpyrrolidone homopolymer.

17. The composition as claimed in claim 16, comprising from 0.1% to 15.0% by weight, preferably from 0.5% to 10.0% by weight and better still from 1.0% to 8.0% by weight of hydrophilic film-forming polymer(s), relative to the total weight of the composition.

18. The composition as claimed in any one of the preceding claims, having at 25° C. a consistency characterized by a hardness≥20 g, preferably ranging from 20 to 200 g, even more preferentially ranging from 30 to 150 g.

19. The composition as claimed in any one of the preceding claims, in the form of an eyelash product such as a mascara, an eyebrow product or a product for the contour of the eyes, such as an eyeliner.

20. An assembly, or kit, for packaging and applying a cosmetic composition for coating keratin fibers, comprising:
a device for packaging comprising the cosmetic composition for coating keratin fibers as defined in any one of the preceding claims;
an applicator for said composition.

21. A process for coating keratin materials, in particular the skin, notably the contour of the eyes, eyelashes or eyebrows, and keratin fibers, such as the eyelashes and/or the eyebrows, comprising a step of applying to said keratin materials at least one composition as defined in any one of claims 1 to 20.
